(19) United States
(12) Patent Application Publication
(10) Pub. No.: US 2006/0165788 A1
(43) Pub. Date: Jul. 27, 2006

(54) Lercanidipine pH dependent pulsatile release compositions

(76) Inventors: Wattanaporn Abramowitz, West Windsor, NJ (US); Ram P. Kapil, Princeton Junction, NJ (US); Todd A. Riccobene, Basking Ridge, NJ (US); Mahendra G. Dedhiya, Pomona, NY (US); Suneel K. Rastogi, Ballwin, MO (US); Anil Chhettry, Holtsville, NY (US)

Correspondence Address: DARBY & DARBY P.C., P. O. BOX 5257, NEW YORK, NY, 10150-5257, US

(21) Appl. No.: 11/223,491

(22) Filed: Sep. 9, 2005

Related U.S. Application Data
(60) Provisional application No. 60/609,222, filed on Sep. 9, 2004.

(51) Int. Cl. A61K 31/445; A61K 9/22

(52) U.S. Cl. 424/468; 514/317

(57) ABSTRACT

Pursuant to the present invention it has been found that a modified release composition containing the low solubility and permeability drug, lercanidipine may be prepared that provides for therapeutically effective plasma concentrations of lercanidipine for 24 hours. The modified release composition of the present invention release pulses of lercanidipine based on the pH of the use environment. An effective quantity of dissolved lercanidipine is released throughout the GI Tract.

### CROSS-REFERENCE TO RELATED APPLICATIONS

The present application claims the benefit of priority under 35 U.S.C. § 119(e) of Provisional Application Ser. No. 60/609,222, filed Sep. 9, 2004, which is hereby incorporated herein by reference in its entirety.

### FIELD OF THE INVENTION

The present invention relates to pulsatile release compositions that achieve release of lercanidipine based on the pH of use environment. The pulsatile release compositions of the present invention deliver lercanidipine with a sustained therapeutic effect. The present invention further provides for unit dosage form compositions comprising spaced multiple pulses based on the pH of the gastrointestinal tract and transit time and may comprise encapsulated beads, granules, or particles or may comprise a tablet with first, second and, optionally, third and/or fourth dosage units. Methods of treatment using the pharmaceutical dosage forms are included within the scope of the present invention.

### BACKGROUND OF THE INVENTION

Modified release dosage forms as defined by the FDA and USP include extended release, delayed release, pulsatile release, and pH dependent release forms and provide a means for improving patient compliance and for ensuring effective and safe therapy by reducing the incidence of adverse drug reactions. Compared to immediate release dosage forms, modified release dosage forms can be used to prolong pharmacologic action after administration, and to reduce variability in the plasma concentration of a drug throughout the dosage interval, eliminating or reducing sharp fluctuations. In light of the advantages of modified release dosage forms, it has been the objective of many skilled in the art to develop such dosage forms.

The majority of modified release dosage forms comprise (1) a core either coated with or containing a drug, wherein the core is further coated with a release modifying layer or (2) a polymeric matrix within which the drug is dispersed and gradually released over time. Both the release modifying layer and the polymeric matrix comprise insoluble or poorly soluble materials that effectively regulate the release of the drug across the layer or through the matrix or provide a burst or extended release upon dissolution of the layer when the composition is exposed to an aqueous environment, i.e. the gastrointestinal (GI) tract. The net rate of release of the drug is dependent on many factors, such as the ability of the gastric fluid to penetrate the coating layer or matrix, the solubility of the drug itself, site specific pH of the GI Tract, and fasted (FA) or fed (FE) conditions.

Because the rate of drug release from a modified release dosage form is dependent in part on the solubility of the drug itself, the development of modified release dosage forms, particularly a pulsatile form with a burst effect, for slightly or poorly soluble drugs (lercanidipine is such a drug) has proven to be more difficult. Therefore, there remains a need in the art for modified release compositions of low solubility drugs and in particular modified release dosage forms containing the poorly soluble drug, lercanidipine, which ensure prolonged therapeutic plasma concentrations of lercanidipine and reduce or eliminate sharp peaks in lercanidipine plasma concentration.

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist with a long duration of action and high vascular selectivity. The molecular formula of the hydrochloride salt of lercanidipine is set forth below.

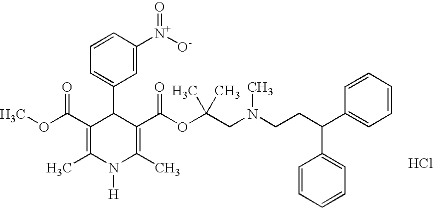

The hydrochloride salt of lercanidipine is commercially available from Recordati S.p.A. (Milan, Italy). Methods of making both lercanidipine free base and its hydrochloride salt have been described previously along with methods of resolving lercanidipine into individual enantiomers in U.S. Pat. Nos. 4,705,797; 5,767,136; 4,968,832; 5,912,351; and 5,696,139, all of which are incorporated herein by reference. Lercanidipine is a dihydropyridine calcium antagonist. As other calcium channel antagonists, it lowers blood pressure by relaxing arteriolar smooth muscle, which decreases peripheral vascular resistance. Lercanidipine produces no negative cardiac inotropism and, occasionally only, mild reflex tachycardia generally of short duration. It has a high affinity for and competitively antagonizes the dihyropyridine subunit of the L-type calcium channel. Lercanidipine has been approved for the treatment of hypertension and has been marketed in several European countries under the trademark Zanidip® since 1996.

Lercanidipine alone or in combination with additional active agents has been shown to be effective in once and twice daily administration. Lercanidipine has been studied in the dosage ranging form 2 to 80 mg. Lercanidipine is normally administered in immediate release tablet form at a dose of about 10 mg to about 20 mg once daily or twice daily. Lercanidipine is used for treating Stage I and Stage II hypertension and is also expected to be useful in alleviating angina pectoris. It has also been beneficial in elderly patients with isolated systolic hypertension. The recommended starting oral dose of lercanidipine is given by mouth 10 mg once daily and is increased, if necessary, after at least 2 weeks to 20 mg daily. Upon oral administration, an immediate release form of lercanidipine is absorbed and peak plasma level Tₘₐₓ occurs 1-3 hours following dosage.

Lercanidipine and its salts, such as the hydrochloride salt, is practically insoluble in water displaying an aqueous solubility of about 5 μg/ml. The solubility of lercanidipine is marginally greater in acidic mediums, however, even at pH 5 it is less than 20 μg/ml. The solubility of lercanidipine at a pH greater than 5 is essentially less than 5 μg/ml. Thus, lercanidipine is essentially insoluble in gastrointestinal pH range of 1 to 8. Lercanidipine is also shows low experimental permeability (i.e., poor permeability, Pₐₐₚ of 0.5×10⁻⁷ cm/s in a Caco-2 cell apparatus and low bioavailability) and is classified as a low permeable drug, as defined by the FDA. Additionally, when administered to patients, lercanidipine displays extensive presystemic first pass elimination as a result of its being a substrate for cytochrome P450 IIIA4 isoenzyme. The combination of poor water solubility, low permeability and considerable first pass metabolism results in low and highly variable bioavailability.

In order to improve the bioavailability of lercanidipine, food is co-administered with each dosage. The administration of food along with lercanidipine has been shown to increase the absorption of lercanidipine significantly and therefore enhance its efficacy, a phenomenon known as “food effect.” Studies have shown that simultaneous intake of food (especially food having a high fat content) increases the amount of lercanidipine absorbed between three and four times compared to administration without food. The same studies have shown that lercanidipine administered in the absence of food is not entirely absorbed which results in low and variable bioavailability. The dependence of effective dosing and absorption of lercanidipine upon co-administration of food is inherently undesirable and can result in fluctuations in effectiveness, inter-patient variability, and in poor patient acceptance and/or compliance.

Accordingly, in order to facilitate the effective administration of lercanidipine alone or in combination with other active agents to patients, there is a need in the art for an oral dosage form which results in absorption and ensures greater bioavailability of lercanidipine particularly therapeutically acceptable minimum plasma concentration (Cₘᵢₙ) is achieved for at least 24 hours for a once-a-day treatment and at the same time a suitable maximum plasma concentration (Cₘₐₓ) is not exceeded. Particularly, there is a need for modified release pharmaceutical compositions that provide modified release of lercanidipine.

### SUMMARY OF THE INVENTION

Pursuant to the present invention it has been found that a modified release composition containing the low solubility and permeability drug, lercanidipine may be prepared that provides for therapeutically effective plasma concentrations of lercanidipine for 24 hours. The modified release composition of the present invention release pulses of lercanidipine based on the pH of the use environment. An effective quantity of dissolved lercanidipine is released throughout the GI Tract.

According to the present invention, the modified release compositions increase Tₘₐₓ thus providing long term plasma concentrations at, the therapeutic plasma concentration.

One embodiment of the present invention provides a pH dependent pulsatile release bead composition comprising multiple pulses of lercanidipine incorporating an immediate release bead containing lercanidipine that substantially releases drug immediately following the exposure to the use environment and a first layer comprising at least one pH dependent release modifying polymer, which dissolves at specified pH following the exposure to the use environment. In one embodiment, the beads are encapsulated. In another embodiment, the beads along with other excipients are compressed to form a tablet.

In one embodiment, the present invention provides a pH dependent pulsatile release tablet compositions comprising multiple pulses of lercanidipine incorporating an immediate release tablet containing lercanidipine that substantially releases drug immediately following the exposure to the use environment and a first layer comprising at least one pH dependent release modifying polymer, which dissolves at specified pH following the exposure to the use environment.

In another embodiment of the present invention the pH dependent pulsatile release bead compositions comprise (i) an immediate release core comprising: (a) an inert core, (b) a first layer comprising a permeability and solubility enhancing surfactant(s), a binder and lercanidipine, and (c) optionally, a second layer comprising a film coating; and (ii) an outer layer comprising: (a) at least one pH dependent release modifying polymer, and (b) optionally, a film coating, wherein the outer layer modifies the release of lercanidipine from the composition at a pH between about 5 and about 6.

In another embodiment of the present invention the pH dependent pulsatile release bead compositions comprise (i) an immediate release core comprising (a) an inert core, (b) a first layer comprising a permeability and solubility enhancing surfactant(s), a binder and lercanidipine, and (c) optionally a second layer comprising a film coating, and (ii) an outer layer comprising (a) at least one pH dependent release modifying polymer, and (b) optionally a film coating, wherein the outer layer modifies the release of lercanidipine from the composition at a pH between about 6 and about 7.

In still another embodiment, the present invention provides a pH dependent pulsatile release composition wherein less than 20% of the lercanidipine is released in pH less than 4.5, simulating fed stomach pH and even less than 10% is released at pH 1.2 simulating fasted stomach pH. In addition, more than about 60% of the lercanidipine is dissolved in vitro within about 180 minutes wherein dissolution is measured by the USP Basket Method 1,100 RPM in 900 ml aqueous buffer containing Polysorbate 80 at 37° C. and pH 5.6, simulating small intestinal pH. In a preferred embodiment, more than about 80% of the lercanidipine is dissolved in vitro within about 180 minutes wherein dissolution of lercanidipine is measured using the USP basket Method 1,100 RPM in 900 ml aqueous buffer containing Polysorbate 80 at 37° C. and pH 6.8, simulating the pH of ileum region of the small intestine.

In an alternative embodiment, the present invention provides a pH dependent pulsatile release composition wherein less than 20% of the lercanidipine is released in pH less than 6, simulating the pH of Duodenum part of the Small Intestinal and even less than 10% is released at pH 1.2 simulating Fasted stomach pH. In addition, more than about 60% of the lercanidipine is released in vitro within about 180 minutes wherein dissolution of lercanidipine is the USP Basket Method 1,100 RPM in 900 ml aqueous buffer containing Polysorbate 80 at 37° C. and pH 6.8, simulating the pH of the ileum region of the small intestine.

In yet another embodiment, the pulsatile release compositions that achieve release of lercanidipine based on the pH of the use environment may be combined to form a unit dosage form having the following characteristics:
(a) A first pulse that has an immediate release composition unit dosage of the active that is released substantially following exposure to pH 1.2 to pH 4.5, simulating stomach pH in fasted and fed state. The first pulse in a preferred embodiment releases more than 80% of the drug in the said pulse unit in at least 60 minutes; and
(b) A second pulse that has a pH dependent compositional unit dosage of the active that is released substantially following exposure to pH greater than about 5.6. The second pulse in a preferred embodiment releases more than 80% of the drug in the said pulse unit in at least 180 minutes. Alternately, the second pulse has a pH dependent compositional unit dosage of the active that is released substantially following exposure to pH greater than about 6.8 simulating small intestine pH. Optionally, the second pulse is a pH dependent compositional unit dosage of the active that is released substantially following exposure to pH greater than about 5.6 simulating pH of the duodenum region. The second pulse in a preferred embodiment releases more than 80% of the drug in the said pulse unit in at least 180 minutes.
(c) A third pulse that has a pH dependent compositional unit dosage of the active that is released substantially following exposure to pH greater than about 6.8 simulating the ileum region. The third pulse in a preferred embodiment releases more than 80% of the drug in the said pulse unit in at least 180 minutes.
(d) A fourth pulse that has a pH dependent compositional unit dosage of the active that is released substantially following exposure to pH greater than about 6.2. This pulse in a preferred embodiment releases more than 80% of the drug in the said pulse unit in at least 180 minutes.
(e) Optionally, the second, third and forth pulses can be such that the active is released substantially at pH 5.6, 6.2 and 6.8

Pursuant to the present invention, administration of the pulsatile release compositions disclosed herein a patient, provides for a rapid increase in lercanidipine plasma concentrations following administration to a peak level from about 8 to about 12 ng/mL, and sustained therapeutic plasma concentration at levels greater than about 0.4 ng/mL for a period of about 20 to 25 hours.

In another embodiment the present invention provides a unit dosage form comprising immediate and pH dependent pulsatile release beads wherein upon administration of the dosage form to a patient the peak plasma concentration of lercanidipine is from about 8 to about 12 ng/mL and the time to peak concentration is from about 2 and 12 hours following administration of the modified bead composition.

In an alternative embodiment, the present invention provides a unit dosage form comprising immediate release and pH dependent pulsatile release beads wherein upon administration of the dosage form to a patient the plasma concentration of lercanidipine is greater than about 0.4 ng/mL from a period of about 20 to about 25 hours after administration.

In still another embodiment, the present invention provides a method of treating a patient suffering from hypertension by administering the pH dependent pulsatile release composition containing lercanidipine disclosed herein, and wherein administration of the composition disclosed herein results in a long term plasma concentration of lercanidipine above therapeutic levels, e.g., plasma concentrations of lercanidipine greater than about 0.1 to about 0.4 ng/ml for a period of about 20 to about 25 hours after administration of the composition of the present invention.

These and other aspects of the present invention will be apparent to those of ordinary skill in the art in the light of the present description, claims and figures.

### DESCRIPTION OF THE DRAWINGS

FIG. 1 depicts the dissolution profile of one pH dependent pulsatile release composition of the present invention (Type I), the curve represented by -▴- is the dissolution profile for modified release composition Type I, cured at 50° C. for 48 hours, the curve represented by -▪- is the dissolution profile for modified release composition Type I, cured at 40° C. and 75% RH for 24 hours, the curve represented by -x- is the dissolution profile for modified release composition Type I, stored at 40° C. and 75% R.H. for 3 months.

FIG. 2 depicts the dissolution profile of one pH dependent pulsatile release composition of the present invention (Type I) in a 2 stage dissolution analysis.

FIG. 3 depicts the dissolution profile of one pH dependent pulsatile release composition of the present invention (Type II), the curve represented by -▴- is the dissolution profile for modified release composition Type II, cured at 50° C. for 48 hours, the curve represented by -▪- is the dissolution profile for modified release composition Type II, cured at 40° C. and 75% RH for 24 hours, the curve represented by -x- is the dissolution profile for modified release composition Type II, stored at 40° C. and 75% R.H. for 3 months.

FIG. 4 depicts the dissolution profile of one pH dependent pulsatile release composition of the present invention (Type II) in a dual dissolution analysis.

FIG. 5 depicts the dissolution profile of one unit dosage form (Prototype I) of the present invention comprising lercanidipine immediate release beads and pH dependent pulsatile release beads Type I, in a dual dissolution analysis.

FIG. 6 depicts the dissolution profile of one unit dosage form (Prototype II) of the present invention comprising lercanidipine immediate release beads and pH dependent pulsatile release beads Type II, in a dual dissolution analysis.

FIG. 7 depicts the dissolution profile of two unit dosage forms (Prototypes VII and IX) of the present invention comprising lercanidipine immediate release beads and a combination of pH dependent pulsatile release beads Type I and Type II, in a three phase dissolution analysis, -♦- is the in vitro dissolution profile for Prototype VII and -▪- is the in vitro dissolution profile for Prototype IX.

FIG. 8 depicts Day 2 in vivo S-lercanidipine plasma concentration, the curve represented by -▴- is the plasma concentration resulting from the administration of 60 mg dosage form (5 mg IR, 25 mg Type I, 30 mg Type II), the curve represented by -▪- is the plasma concentration resulting from the administration of 60 mg dosage form (5 mg IR, 55 mg Type I), and curve represented by -●- is the plasma concentration resulting from the administration of 30 mg dosage form (5 mg IR, 25 mg Type I).

FIG. 9 depicts Day 7 in vivo S-lercanidipine plasma concentration, the curve represented by -▴- is the plasma concentration resulting from the administration of 60 mg dosage form (5 mg IR, 25 mg Type I, 30 mg Type II), the curve represented by -▪- is the plasma concentration resulting from the administration of 60 mg dosage form (5 mg IR, 55 mg Type I), and curve represented by -●- is the plasma concentration resulting from the administration of 30 mg dosage form (5 mg IR, 25 mg Type I).

### DETAILED DESCRIPTION OF THE INVENTION

As used herein, the following terms are defined as follows:

The term “about” means within 10% of a given value, preferably within 5%, and more preferably within 1% of a given value. Alternatively, the term “about” means that a value can fall within a scientifically acceptable error range for that type of value, which will depend on how qualitative a measurement can be given the available tools.

The phrase “dissolution profile” as used herein, refers to the dissolution of an agent over time. The dissolution can be measured as relative amount agent dissolved over time, the amount of agent dissolved, or the concentration of the agent.

The term “lercanidipine” means the free base composition methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate, as well as any pharmaceutically acceptable salt, e.g., a salt with an inorganic or organic acid such as, HCl, HBr, H₂SO₄, maleic acid, fumaric acid, tartaric acid and citric acid. Preferred pharmaceutically acceptable salts of lercanidipine include, but are not limited to, hydrochloride, besylate and napadisylate salts. Additionally, lercanidipine may be present in crystalline and/or amorphous forms. Preferred pharmaceutically acceptable salts of lercanidipine include may be either R or S enantiomers, or a racemic mixture thereof.

The term “modified release” means any type of release of the active ingredient, lercanidipine, from the composition of the present invention resulting in modified release over a period of time sufficient to maintain therapeutically effective plasma levels over similarly extended time intervals and/or to modify other pharmacokinetic properties of the active ingredient. Preferably the release provides for therapeutic plasma concentrations of lercanidipine for a period for about 20 to about 25 and an average plasma concentration of lercanidipine of at least about 0.1 to about 0.4 ng/mL over the duration of the dosing interval.

The term “pH dependent” means a composition having characteristics which vary according to environmental pH, e.g., due to pH changes in the in vitro dissolution media or due to passage of the dosage from through the gastrointestinal tract.

The term “bioavailability” refers to the rate and extent to which the active ingredient or active moiety, e.g., lercanidipine, is absorbed from a drug product, i.e., bead, and becomes systematically available.

As used herein, the term “pharmaceutically acceptable” refers to a biologically or pharmacologically compatible for in vivo use, and preferably means approved by a regulatory agency of the Federal or a state government or listed in the U.S. Pharmacopoeia or other generally recognized pharmacopoeia for use in animals, and more particularly in humans.

The terms “treat” and “treating” refer to reducing or relieving hypertension, e.g., decreasing either systolic or diastolic blood pressure in a patient by at least 10 mm Hg.

As used herein, a “therapeutically effective amount” refers to the amount of an active agent sufficient to lower the blood pressure of a patient with hypertension, e.g., the blood pressure is decreased by at least about 15 mm Hg for systolic pressure or by about 10 mm Hg for diastolic pressure. A therapeutically effective amount of the active agent may or may not decrease the blood pressure in a person that does not have hypertension or may not decrease blood pressure in all persons with hypertension. In a preferred embodiment, the active agent decreases a patient's blood pressure to below 140/90 mm Hg. Treatment of other pathologies, such as heart failure or arthrosclerosis is also specifically contemplated as per, e.g., U.S. Pat. Nos. 5,696,139 and 5,767,136.

All weights and weight ratios specified for lercanidipine and pharmaceutically acceptable salts thereof are based on the weight of a molar equivalent of the hydrochloride salt of lercanidipine.

Pharmaceutical Compositions

The pH dependent pulsatile release compositions of the present invention is designed to provide pH dependent release of lercanidipine upon exposure of the composition to a use environment, e.g. (1) the fluid in the lower intestine or colon, having a pH greater than that of the stomach fluid, i.e., greater then about 5 or (2) at pH greater than about 1.2. Moreover, it has been discovered that the compositions of the present invention provide for extended release of lercanidipine in vivo over extended durations, i.e., for a period of about 20 to about 25 hours compared to prior art formulations.

The pH dependent pulsatile release compositions of the present invention comprise an immediate release core containing active substantially enveloped by at least one pH dependent release modifying polymer and optionally a film coating. The pH dependent release modifying polymer is intended to direct the release of lercanidipine to an use environment e.g., having a pH greater than that of gastric fluid, i.e., pH greater than 1.2, preferably more than about 5 and to facilitate the pulsatile release of lercanidipine from the immediate release core, thereby providing for long term therapeutic levels of lercanidipine when the composition is administered to a patient.

Immediate Release Core

In one embodiment, the inert core may comprise any pharmaceutically acceptable material, including but not limited to inorganic or organic non-pareil seeds, such as those made from microcrystalline cellulose, Sucrose (sugar) or starch. Preferably the inert core has a mean size from about 10 and about 80 mesh. Preferably the ratio of the mass of the inert core to the mass of lercanidipine is from about 5:1 to about 20:1 and more preferably from about 5:1 to about 15:1.

The inert core is coated with a first layer comprising lercanidipine, a solubility/permeability enhancing surfactant and a binder. In one preferred embodiment, the lercanidipine is lercanidipine hydrochloride. Additionally, lercanidipine may be present in either crystalline or amorphous forms and in one or both of its enantiomeric forms. Lercanidipine which is present in the crystalline form may be present in any polymorphic form or mixtures thereof, including those disclosed in U.S. Published Application Nos. 2003/0083355 and 2003/0069285 which are incorporated herein by reference. Preferred pharmaceutically acceptable polymorphs of lercanidipine are crystalline Forms I and II. Additionally, lercanidipine may be amorphous or a mixture or amorphous and crystalline, wherein the crystalline can be of the same polymorph or a combination of two or more polymorphs.

One skilled in the art will appreciate that the pulsatile release compositions of the present invention may include one or more forms of lercanidipine, e.g., different salt forms, amorphous forms or crystalline forms, in order to achieve the desired in vitro dissolution profile and/or the desired in vivo plasma concentration of lercanidipine. In one embodiment, one skilled in the art may combine crystalline lercanidipine forms (I) and (II) to achieve desired properties, based upon bioavailability studies described in US. Published Application 2003/0083355 (herein incorporated by reference) that found lercanidipine crystalline polymorph form (II) to have a higher bioavailability than lercanidipine crystalline polymorph Form (I). Studies have also indicated, however, that form (I) has a shorter time to maximum concentration attainable compared to form (II) and that form (II) has a higher plasma concentration (AUCo-t) and a delayed time of maximum concentration (Tₘₐₓ), compared to Form (I). The novel present invention incorporates sufficient solubility/permeability enhancer surfactant that allows for the use of different polymorphs.

Preferably lercanidipine is present in an amount sufficient to render a therapeutic effect when the modified release composition of the present invention is administered to a patient. Lercanidipine may be present any amount from about 0.001 to about 0.3 mg per mg of the total composition, and more preferably from about 0.005 mg to about 0.2 mg per mg of the total composition and most preferably 0.01 mg about 0.15 mg per mg of the total composition.

In addition to lercanidipine, the first layer coating the inert core comprises a surfactant. Surfactants may be incorporated in the beads of the present invention to facilitate the wetting of lercanidipine, increased dissolution, and permeation of lercanidipine in the environment of use. Surfactant may also be incorporated for the purpose of enhancing or modulating the solubility of lercanidipine.

Surfactants of the present invention include, but are not limited to anionic and non-ionic surfactants such as sodium lauryl sulfate, poloxamers (copolymers of polyoxyethylene and polyoxypropylene), natural or synthetic lecitins as well as esters of sorbitan and fatty acids, such as Span® (commercially available from Sigma-Aldrich Co., St. Louis, Mo.), esters of polyoxyethylenesorbitan and fatty acids, such as Polysorbates or Tween® (commercially available from Spectrum, CA or Sigma-Aldrich Co.) polyoxyethylated hydrogenated castor oils, such as Cremophor® (commercially available from BASF, Mount Olive, N.J.), polyoxyethylene stearates, such as Myrj® (commercially available from Uniqema, New Castle, Del.) or any combinations of the said surfactants. Preferably the surfactant is a polysorbate and most preferably the surfactant is Polysorbate 80 (e.g., Tween® 80, commercially available from Sigma-Aldrich Co., St. Louis, Mo.) or Vitamin TPGS (Eastman Chemical, Kingsport, Tenn.).

The amount of surfactant may be adjusted, so as to moderate the solubility, permeability and bioavailability of lercanidipine. Preferably the ratio of surfactant to lercanidipine on a mass basis is from about 0.001:1 to about 0.2:1, more preferably from about 0.005:1 to 0.1:1 and most preferably from about 0.01:1 to about 0.075:1.

The first layer coating the inert core further comprises a binder. Binders are incorporated in the beads of the present invention to facilitate the adhesion of lercanidipine to the inert core. Preferably, the binder does not interfere with or decrease the solubility of lercanidipine. Suitable binders include, but are not limited to, either individually or in combination, such binding agents and adhesives as sucrose; gelatin; glucose; starch; cellulose materials such as, but not limited to, methylcellulose and sodium carboxymethylcellulose; alginic acid and salts of alginic acid; magnesium aluminum silicate; polyethylene glycol; guar gum; polysaccharide acids; bentonites; polyvinylpyrrolidone (povidone); polymethacrylates; hydroxypropyl methylcellulose (HPMC); hydroxypropyl cellulose (Klucel™); ethyl cellulose (Ethocel™); pregelatinized starch (such as National™ 1511 and Starch 1500).

Preferably the binder comprises hydroxypropylmethyl cellulose and most preferably Opadry™ Clear (commercially available from Colorcon, Inc., West Point, Pa.). Preferably the ratio of binder to lercanidipine on a mass basis is from about 0.01:1 to about 1:1, more preferably from about 0.05:1 to 0.5:1 and most preferably from about 0.1:1 to about 0.3:1.

Optionally the immediate release core may comprise a second layer comprising a film coating to improve the durability, appearance and/or handling of the bead composition. Preferably the film coating does not interfere with the dissolution and/or pharmacokinetic properties of the composition of the present invention. Examples of film coatings contemplated by the present invention include, but are not limited to, those that include hydroxypropylmethyl cellulose and particularly Opadry™, Eudragits™, and PVP. However, any film-former known in the art may be used. The film coating is to be applied to the immediate release core, the preferred ratio of film coating to lercanidipine is from about 0.01:1 to about 1:1, more preferably from about 0.03:1 to 0.5:1 and most preferably from about 0.05:1 to about 0.3:1. The optional coating also minimizes migration of lercanidipine into the subsequent modifying polymer layer. One skilled in the art will appreciate that the rate of lercanidipine release from the composition may be affected by migration of lercanidipine into subsequent pH dependent polymer layer. Optionally, one skilled in the art will appreciate the core can be compressed into a tablet or filled in to capsules for subsequent coating applications.

Coating Immediate Release Compositions to Yield pH Dependent Pulsatile Release Bead

In one embodiment, the immediate release core may be coated with a pH dependent release modifying polymer to create a pH dependent pulsatile release bead. The modifying polymer coating is intended to release lercanidipine from the first layer at pH dependent release in the gastrointestinal tract, thereby providing the desired extended in vitro release rate or in vivo plasma concentrations of lercanidipine. Moreover, the release modifying polymer is intended to facilitate the release of lercanidipine in the preferred environmental fluid, e.g., the fluid of the small intestine or the colon.

In addition to regulating the release of lercanidipine, the pH dependent release modifying polymer should be capable of producing a strong, continuous film that is smooth and elegant, capable of supporting pigments and other coating additives, inert and tack-free.

In one embodiment of the present invention, the pH dependent pulsatile release bead composition comprises an immediate release core and a subsequent layer comprising a pH dependent release modifying polymer. In one embodiment of the present invention the pH dependent release modifying polymer is relatively insoluble and impermeable at the pH of the gastric fluid in the stomach, but is substantially soluble and permeable at the elevated pH of the small intestine or colon. Additionally, the pH dependent bead compositions can be further coated with additional immediate release and/or pH dependent polymers there by creating multiple pulses from a unitary bead composition.

pH dependent release modifying polymers contemplated by the present invention include, but are not limited to polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Preferred pH dependent release modifying polymers include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

Most preferably the pH dependent release modifying polymer of the present invention is an anionic acrylic copolymers of methacrylic acid, methylmethacrylate, and methacrylic acid co-polymer type C. Such co-polymers are particularly useful coating materials for delaying the release of lercanidipine from the modified release beads of the present invention until the beads have passed through the stomach and to an use environment having a pH greater then that of the gastric fluid, i.e., the fluid of the lower intestine or colon. Anionic acrylic copolymers of methacrylic acid and methylmethacrylate, contemplated by the present invention are commercially available from many suppliers. One skilled in the art will appreciate that these are supplied by companies such as Röhm Pharma GmbH (Weiterstat, Germany) under the tradenames Eudragit-L® Eudragit-S® Eudragit FS®, Colorcon, Inc. (West Point, Pa.) under the tradename Acryl-Eze®, Eastman Chemical (Kingsport Tenn.) under the trade name Eastacryl® 30 D and under many and generic brand names. In addition, alternate polymers supplied by Eastman Chemical (Kingsport Tenn.) (CAP, known as cellulose acetate phthalate), Colorcon, Inc. (West Point, Pa.) under the trade name Sureteric® and under many and generic brand names can also be used.

Eudragit-L® and Eudragit-S® are anionic copolymers of methacrylic acid and methylmethacrylate. The ratio of free carboxyl groups to the esters is approximately 1:1 in Eudragit-L® and approximately 1:2 in Eudragit-S®. Because of the difference in the ratio of free carboxyl groups to the esters Eudragit-L® and Eudragit-S® are soluble at different pH levels. For example Eudragit-L® films dissolve about above pH 5.5 Eudragit-L® 30D and Eudragit-L® 100-55 (methacrylic acid co-polymer type C, USP/NF); about above pH 6, Eudragit-L® 100 and (methacrylic acid co-polymer type A, USP/NF); and Eudragit-S® 100 and Eudragit-FS® 30 D (methacrylic acid co-polymer type B, USP/NF) films dissolve about above pH 7.0. Acryl-Eze® contains methacrylic acid co-polymer type C, USP/NF which is similar to the Eudragit L100-55.

One skilled in art will appreciate that film dissolution pH's are also affected by several factors, such as ionic strengths, lipid contents, pigments, plasticizers, etc.

The present inventions embody the use of Eudragit-L® and Eudragit-S® separately or in combination in the outer layer of the modified release composition disclosed herein. In one embodiment, the release of lercanidipine may be controlled by choice of both Eudragit-L® and Eudragit-S® or combinations of thereof in the coating. Mixtures of Eudragit-L® and Eudragit-S® dissolve at pHs between about 6 and about 7. Since the pH of the small intestine ranges from about 5 to about 7, e.g., duodenum is approximately 6.0 and the pH of the colon is approximately 7.0, outer layers composed of mixtures of Eudragit-L® and Eudragit-S® provide for release of lercanidipine selectively in either the fluid of the small intestine or the colon. If it is desired to delay release of lercanidipine until the lercanidipine-containing bead composition has reached the colon, Eudragit-S® may be used as the coating material, as described by Dew et al. (Br. J. Clin. Pharmac. 14 (1982) 405-408) or Eudragit-FS® may also be used.

In one embodiment, in order to initiate the release of lercanidipine in a pulsatile burst in about the first 30 minutes, after the modified release composition has exited the stomach and ileum, and to ensure dissolution of the outer coating at a pH of about 6.5 a combination of Eudragit-L® and Eudragit-S® may be employed. One outer layer composition comprise from about 9:1 to about 1:9 Eudragit-L®/Eudragit-S®, more preferably from about 9:1 to about 1:4 Eudragit-L®/Eudragit-S®. The outer layer may comprise between about 3% and about 70% of the weight of the composition. Preferably, the ratio of the mass of the outer layer to the mass of lercanidipine is from about 2:1 to about 0.05:1 and more preferably from about 1:1 to about 0.1:1 and still more preferably from about 0.7:1 to about 0.3:1.

In an additional embodiment the release of lercanidipine may be modified to occur at above pH of about 5 and more preferably above about 5.5 by coating the composition with an outer layer of methacrylic acid co-polymer type C (Acryl-Eze®). As will be appreciated by one skilled in the art, the amount and thickness of Acryl-Eze® in the outer layer of the modified release bead composition may be varied to obtain the desired release profile of lercanidipine. Preferably, the ratio of the mass of the outer layer to the mass of lercanidipine is from about 2:1 to about 0.05:1 and more preferably from about 1:1 to about 0.1:1 and still more preferably from about 0.6:1 to about 0.3:1.

Preferably the amount of outer layer is applied in an amount sufficient to yield a modified release bead having the desired dissolution profile and/or pharmacokinetic profile. Most preferably the outer layer is applied, such that the beads have an average radius from about 10 mesh to about 140 mesh mm and most preferably from about 14 mesh to about 60 mesh mm. Optionally, an inert core with a mean size from about 35 and about 140 mesh, can also be used for compression of beads into tablets.

One skilled in the art will appreciate that the rate of lercanidipine release from the modified release bead composition may be controlled by factors such as the composition, surfactant, and binder content of the immediate release core; the thickness and permeability of the release modifying acrylic polymer coating; and the surface area-to-volume ratio of the beads themselves. It will be appreciated by those skilled in the art that increasing the thickness of the coating will decrease the release rate, whereas increasing the permeability of the coating or the surface area-to-volume ratio of the beads will increase the release rate. One skilled in the art will appreciate that the above mentioned factors may be adjusted such that the modified release composition of the present invention achieves the desired in vitro dissolution rate, and/or the in vivo plasma concentration of lercanidipine over time. Moreover, the optional coating level for the core may affect the release rate of the lercanidipine.

Moreover, it will be appreciated by those skilled in the art that the desired in vitro dissolution rate, and/or the in vivo plasma concentration of lercanidipine over time, may be obtained by selecting one or more forms of lercanidipine, i.e., selecting one or more salts forms, crystalline forms (including one or more polymorphic forms), amorphous forms, and enantiomeric forms for use in the modified release beads of the present invention.

In another preferred embodiment of the present invention where the outer layer comprises an acrylic polymer, an effective amount of a plasticizer may be included to improve the physical properties of the outer layer. Suitable plasticizers embodied by the present include, but are not limited to, citric acid esters such as triethyl citrate, tributyl citrate, dibutyl phthalate, and possibly 1,2-propylene glycol, polyethylene glycols, propylene glycol, diethyl phthalate, castor oil, and triacetin. In one preferred embodiment the acrylic polymer forming the outer layer includes triethyl citrate as a plasticizer.

Plasticizers may be incorporated into the outer layer in any amount sufficient to impart the sustained release composition of the present invention with the desired physical properties. Preferably the plasticizer is present in amount between about 5 and about 15% w/w of the polymer and most preferably between about 8 and about 10%. One skilled in the art, however, will appreciate that the precise amount of plasticizer may depend upon several factors including the type of polymer and the coating conditions.

Optionally, the pharmaceutical compositions of the present invention may include additional excipients to improve appearance, handling and processing properties and/or dissolution properties of the active ingredient. Additional excipients contemplated by the present invention include, but are not limited to, carriers, dilutents, lubricants, glidants and/or anti-adherent agents.

Suitable lubricants and/or glidants include, but are not limited to, either individually or in combination, such lubricants and/or glidants as glyceryl behenate (Compritol™ 888); metallic stearates (e.g., magnesium, calcium and sodium stearates); stearic acid; hydrogenated vegetable oils (e.g., Sterotex™); talc; waxes; Stearowet™; boric acid; sodium benzoate and sodium acetate; sodium chloride; DL-Leucine; polyethylene glycols (e.g., Carbowax™ 4000 and Carbowax™ 6000); sodium oleate; sodium benzoate; sodium acetate; sodium lauryl sulfate; sodium stearyl fumarate (Pruv™); and magnesium lauryl sulfate.

Additional suitable anti-adherents or glidants include, but are not limited to, either individually or in combination, such anti-adherents as talc, cornstarch, DL-Leucine, sodium lauryl sulfate, and metallic stearates.

Other carrier materials (such as colorants, flavors and sweeteners) and modes of administration are known in the pharmaceutical art and can be used in the preparation of the pharmaceutical compositions of the present invention.

Manufacture of Pharmaceutical Compositions

The pH dependent pulsatile beads of the present invention may be manufactured using any number of processes well known in the art. In one embodiment the composition of the present invention may be prepared as a bead by first forming an immediate release core by coating an inert core with an aqueous suspension containing lercanidipine followed by optionally an additional layer of Opadry™. The immediate release core may then be coated with an outer coating comprising a release modifying acrylic polymer to prepare the modified release composition of the present invention. Optionally, a film coating may be applied over the release modifying acrylic polymer to enhance the durability and appearance of the bead.

In one embodiment, inert cores are preheated in a fluidized bed coater (e.g., GPGC5, Glatt Air Technique, Ramsey N.J.), for about 10 minutes and more preferably for about 5 minutes, between about 30° C. and about 45° C. and more preferably between about 35° C. and 40° C. Drug loading may be carried out using any method known in the art, such spray coating, although other coating methods may be used. Preferably, the inert cores are coated with a suspension containing lercanidipine, a binder, a surfactant and purified water in a fluidized bed coater using a spray pressure between about 1 and 3 bars, at a temperature between 30° C. and about 45° C. and more preferably between about 36° C. and 42° C.

Drug loaded beads may be film coated by coating the beads with an aqueous dispersion of material such as Opadry™. An aqueous film coating dispersion may be applied using any method known in the art, such as spray coating the beads in a fluidized bed coater at a spray pressure between about 1 and 3 bars and a temperature between about 35° C. and about 55° C. and more preferably between about 40° C. and 50° C.

An aqueous suspension containing the pH dependent release modifying polymer may be applied to the immediate release core by spraying, using any suitable spray equipment known in the art. An aqueous suspension containing the pH dependent release modifying polymer may be prepared by dissolving the polymer in water or in an organic solvent or mixture of organic solvents. Useful organic solvents for this purpose are acetone, isopropyl alcohol, and methylene chloride. The aqueous suspension by also include a plasticizer. Useful plasticizers include citric acid esters such as triethyl citrate, tributyl citrate, dibutyl phthalate, and possibly 1,2-propylene glycol, polyethylene glycols, propylene glycol, diethyl phthalate, castor oil, and triacetin. The plasticizer may be present in an amount from about 8 to about 10% based upon the total weight of polymers in the composition.

The bead compositions of the present invention may be cured and dried following manufacture. One skilled in the art will appreciate that drying and curing conditions will vary depending upon several factors including for example, the size of the substrate, the thickness of the coating, and the amount of hydrophobic material in the composition. In one embodiment, the immediate release cores are dried in a fluidized bed for about 10 minutes and more preferably for about 5 minutes, between about 30° C. and about 45° C. and more preferably between about 35° C. and 40° C. Optionally, the modified release compositions of the present invention are cured in an oven or other suitable devise at about between 40° C. and 60° C. and more preferably about 50° C. for between about 4 and about 48 hours.

Unit Dosage Forms

To form oral unit dosage forms, dried beads may be combined and loaded into gelatin capsules, or other delivery devices suitable for oral administration or compressed into tablets. Preferably the unit dosage forms comprise a sufficient amount of the modified release beads of the present invention to impart a therapeutic effect when the dosage form is administered to a patient. More preferably the unit dosage form comprises from about 2 to about 80 mg of lercanidipine, and most preferably about 5 to about 80 mg of lercanidipine.

In one embodiment, the modified release compositions of the present invention may be combined with an immediate release composition and/or another modified release composition to yield a unit dosage exhibiting a multi-phase release profile at least one of which is a pH dependent composition. In another embodiment, the pH dependent pulsatile the unit dosage form comprises both immediate and pH dependent release compositions and provides a total dosage of lercanidipine of about 2 to about 80 mg of and more preferably about 5 to about 80 mg, wherein from about 4.5 to about 75 mg of the lercanidipine is provided as a modified release composition.

Preferably the ratio of immediate release to pH dependent pulsatile release compositions is such that the dosage form, when administered to a patient, provides both rapid and longer term relief from hypertension. Preferably, the ratio of immediate to modified release compositions is such that the dosage form provide for Tₘₐₓ from about 3 to about 12 hours, maximum plasma concentration of lercanidipine from about 10 to about 14 ng/mL, and therapeutic plasma concentrations of lercanidipine for a period for about 20 to about 25 hours. In one embodiment the ratio of immediate release to modified release compositions is preferably from about 1:1 and 1:50, more preferably from about 1:2 and 1:20 and most preferably from about 1:5 and 1:15.

Dissolution Profile

The modified release compositions of the present invention are designed to provide modified release of lercanidipine over the duration of the dosing interval. To ensure that the modified release compositions provide the desired effect in vitro, e.g., therapeutic plasma concentrations for a period of about 20 to about 25 following administration to a patient, it is first necessary to establish desired in vitro dissolution properties. The presence of a dissolved substance in the gastrointestinal fluid and subsequent permeation of the drug are essential to ensure sufficient bioavailability, therefore dissolution properties of an active agent are important in evaluating its ability to be absorbed and made available at the site of action. Therefore, when evaluating the potential bioavailability of an active agent, it is important to determine its dissolution profile.

The dissolution profile for an active agent from a dosage unit is determined as the proportion of the amount of active agent released from the dosage unit over a specified time. The test method used references the results, so it is important to specify the method as well as the conditions under which measurements were made. Preferably the dissolution properties of the pH dependent pulsatile release compositions of the present invention are determined using the dissolution method, USP basket method at 100 RPM in 900 ml aqueous buffer having a pH between about 1.2 and about 7.0, at 37° C. Alternate methods such as those described in the USP, e.g., paddle method at 50 RPM in 900 ml aqueous buffer may be acceptable.

With the above in mind, in one embodiment the in vitro dissolution of lercanidipine at various time points for compositions in accordance with the present invention is preferably pulsatile with about 50% dissolved within about 1 hour, and more preferably at least about 80% dissolved within about 180 minutes. In addition, the undesired leaching of the active drug for modified pH dependent release composition should be less than 20% at specified lower pH. For example, the composition coated with methacrylic acid co-polymer type C should not release more than 20% of the drug in stomach pH of 1.2 whereas the composition coated with methacrylic acid co-polymer type B should not release more than 20% of the drug from pH of about 1.2 to 6.

Pharmacokinetic Profiles

In addition to providing for pulsatile release of lercanidipine in an use environment, e.g., the fluid of the small intestine or colon, it is an objective of the present invention to provide a pH dependent pulsatile release composition having a pharmacokinetic profile which provides for sustained of relief of symptoms associated with hypertension, while avoiding undesirable side effects. Such a pharmacokinetic profile provides for a gradual rise in lercanidipine plasma concentration to therapeutic levels following administration to a patient, e.g., from about 8 to about 12 ng/ml of lercanidipine, followed by a steady decline in plasma concentration to a level from about 0.1 to 0.4 ng/ml of lercanidipine. Preferably, the pharmacokinetic profile does not have any erratic peaks or troughs, but rather provides for a steady and consistent rise in lercanidipine concentration to therapeutic levels, followed by a steady and consistent decline.

Additionally, it is an objective of the present invention to provide modified release composition which provides for sustained, e.g., long term, plasma concentration of lercanidipine at therapeutic levels. Preferably, upon administration of the modified release composition of the present invention to a patient the composition provides for sustained therapeutic plasma concentrations of lercanidipine for about 20 to about 25 hours.

Treatment of Specific Conditions and Disorders

The pharmaceutical composition or unit dosage forms of the present invention may be administered to an animal, preferably a human being, in need of anti-hypertensive treatment. The pharmaceutical composition or unit dosage form of the present invention may be administered according to a dosage and administration regimen defined by routine testing in light of the guidelines given above in order to obtain optimal antihypertensive activity and a decrease in blood pressure while minimizing toxicity or side-effects for a particular patient. However, such fine turning of the therapeutic regimen is routine in light of the guidelines given herein.

The dosage of the compositions of the present invention may vary according to a variety of factors such as underlying disease state, the individual's condition, weight, sex and age and the mode of administration. For oral administration, the pharmaceutical compositions can be provided in the form of scored or unscored unit dosage forms.

In a preferred embodiment, for the treatment of hypertension, a patient may be administered the pH dependent pulsatile release composition or dosage form, wherein the total dosage of lercanidipine is from about 2 to 80 mg. More preferably, the composition or dosage form comprises from about 5 to 80 mg lercanidipine.

The pharmaceutical composition or unit dosage form may be administered in a single daily dose, or the total daily dosage may be administered in divided doses. In addition, co-administration or sequential administration of other active agents may be desirable. The pH dependent pulsatile release bead compositions of the invention may be combined with any known drug therapy, preferably for treatment of hypertension. For example, bimodal therapy involving in addition a diuretic, a β-receptor blocker, an ACE inhibitor or an angiotensin II receptor antagonist is contemplated by the present invention (see, e.g., U.S. patent application Ser. No. 10/791,148, which is hereby incorporated by reference).

The lercanidipine formulation of the current invention may be combined with additional active agents. Two different 1,4-dihydropyridines may be used, or the lercanidipine may be combined with other active agents or other therapies For example, a lercanidipine formulation may be combined with an ACE inhibitor, such as enalapril, described in U.S. Patent Publication No. 2003/0180355, or with lisinopril as described in commonly-owned U.S. patent application Ser. Nos. 10/688,061 and 10/829,932. Lercanidipine may also be combined with an angiotensin II receptor blocker (ARB) such as irbesartan or olmesartan (U.S. patent application Ser. No. 10/791,148). Also contemplated by the present invention is addition of a diuretic or a receptor blocker to the lercanidipine formulation. Exemplary diuretics include thiazide diuretics, potassium sparing diuretics, or loop diuretics, such as hydrochlorothiazide, spironolactone, and ethacrynic acid, respectively.

Non-limiting examples of ACE inhibitors include benazepril, captopril, cilazapril, enalapril, fentiapril, fosinopril, indopril, lisonopril moexipril, perindopril, quinapril, ramipril, spirapril and their pharmaceutically acceptable salt Exemplarily diuretics include furosemide, hydrochlorothiazide, torasemide, indapamide and eplerenone. Exemplarily CCB's include amlodipine, nifedipine and verapamil. Exemplarily beta blockers include atenolol, carvediol, nadolol and propranolol. Exemplarily alpha blockers include clonidine and prazosin. Exemplarily ARBs include, candesartan, eprosartan, irbesartan, losartan, olmesartan, saprisartan, telmisartan, valsartan, E-1477, SC-52458, ZD-8731, and their pharmaceutically acceptable salts. Additional ARBS include those found in EP 443983, EP 253310, EP 403159, EP 420237, EP 502314, EP 504888, EP 514198, EP 475206, and WO 93/20816, each of which are herein incorporated by reference.

The lercanidipine formulations may also be combined in a therapy with a second active agent, such as those described above, where the two agents are administered sequentially. Either the lercanidipine or the second agent may be delivered first, and the time between treatment of the lercanidipine and second agent may be for a period from about 1-2 hours, to about 2-6 hours, to about 6-12 hours, to about 12-24 hours following administration of the first agent. Similarly, this same time period may occur between a first and third agent in the case of a three-way combination. Alternatively, simultaneous administration of the 1,4-dihydropyrine and second active agent, with or without sequential administration of either the 1,4-dihydropyrine and second active agent could also be employed.

For combination therapy the compounds may initially be provided as separate dosage forms until an optimum dosage combination and administration regimen is achieved. Therefore, the patient may be titrated to the appropriate dosages for his/her particular hypertensive condition. After the appropriate dosage of each of the compounds is determined to achieve a decrease of the blood pressure without untoward side effects, the patient then may be switched to a single dosage form containing the appropriate dosages of each of the active agents, or may continue with a triple dosage form.

The exact dosage and administration regimen utilizing the combination therapy of the present invention is selected in accordance with a variety of factors including type, species, age, weight, sex and medical condition of the patient; the severity and etiology of the hypertension to be treated; the route of administration; the renal and hepatic function of the patient; the treatment history of the patient; and the responsiveness of the patient. Optimal precision in achieving concentrations of compounds within the range that yields efficacy without toxicity requires a regimen based on the kinetics of the drug's availability to target sites. This involves a consideration of the absorption, distribution, metabolism, excretion of a drug, and responsiveness of the patient to the dosage regimen. However, such fine tuning of the therapeutic regimen is routine in light of the guidelines given herein.

In a preferred embodiment of the present invention, the composition is administered daily to the patient. In a further preferred embodiment, the daily pharmaceutical composition or dosage form comprises 0.1 to 80 mg lercanidipine. Preferably, the daily composition or dosage form comprises 2 to 80 mg lercanidipine. More preferably, the daily composition or dosage form comprises 5 to 80 mg lercanidipine.

### EXAMPLES

The following examples of immediate release pharmaceutical bead compositions and methods of making the same are now disclosed. The following examples are illustrative in nature of the various aspects of the present invention and are not intended to be limiting in any manner.

### Example 1

### Preparation of Lercanidipine Immediate Release Core

The present examples describe the composition and manufacture of an immediate release core. The composition of the immediate release core is shown in Table 1 below. All weights are provided on the basis of the mass of the dried bead composition.

TABLE 1
Lercanidipine immediate release cores

| Ingredient | mg/g | Weight % Composition |
|---|---|---|
| Lercanidipine HCl | 122.6 | 12.26 |
| Polysorbate 80, NF | 9.2 | 0.92 |
| Sugar Spheres, USP | 818 | 81.80 |
| Opadry Clear (Binder) | 30.6 | 3.06 |
| Opadry Clear (Film | 19.6 | 1.96 |
| Coating) | | |

The lercanidipine immediate release core of the present example was prepared by loading approximately 8.18 kg sugar spheres, USP Paular Crop, Cranbury, N.J. having a size of approximately 20-25 mesh into a GPCG5 fluidized bed coater. The sugar spheres were preheated for about 5 minutes between 34 and 44° C.

The preheated spheres were spray coated with an aqueous lercanidipine suspension in a GPCG5 fluidized bed coater, using a Wuster Sytem Glatt Air Technique, Ramsey, N.J. at a spraying pressure between 1 and 3 bars and a temperature between 34 and 44° C.

The lercanidipine suspension was prepared by first preparing a suspension of Opadry™ Clear by mixing 0.306 Kg Opadry™ Clear (Colorcon, Inc., West Point, Pa.) in 11.6 L purified water with continuous stirring until fully dissolved. The suspension of Opadry™ Clear was divided into equal halves. To one half 0.092 Kg Polysorbate 80 (Spectrum Chemical, New Brunswick, N.J. was added with continuous stirring followed by the addition of 1.226 Kg lercanidipine HCl (Recordati SpA, Milan, Italy). Once the lercanidipine HCl was fully dispersed, the second half of the Opadry™ Clear was added to complete the solution.

Following drug loading the beads were film coated by coating with Opadry™ Clear YS-1-7006. Dispersion of Opadry™ Clear was prepared by mixing 0.196 Kg Opadry™ Clear with 2.45 L purified water with continuous stirring until the Opadry™ Clear was completely dissolved. The film coating solution was applied by spraying the beads in a fluidized bed coater using a spray pressure between about 1 and 3 bars, at a product temperature between about 34 and 44° C.

Film coated beads were dried in a fluidized bed for about 5 minutes between about 34 and 44° C. Optionally, multiple sub lots of beads were mixed in a V-blender and stored sealed under suitable conditions.

### Example 2

### Preparation of Type I pH Dependent Pulsatile Release Beads

The present example describes the composition and manufacture of a lercanidipine pH dependent pulsatile release bead in which methacrylic acid co-polymer type C (Acryl-Eze®) is applied as an outer coating member to the immediate release core described in Example 1. The composition of the modified release bead of the present Example is shown in Table 2 below. All weights are provided on the basis of the mass of the total mass of the final encapsulated dosage form.

TABLE 2
Type I, lercanidipine pH dependent pulsatile release bead

| Ingredient | mg/capsule | Weight % Composition |
|---|---|---|
| Lercanidipine immediate | 163.2 | 76.9 |
| release core (122.55 mg/g) | | |
| Methacrylic acid co-polymer | 48.96 | 23.1 |
| type C (Acryl-Eze ®) | | |

A fraction of the immediate release cores, prepared as described in Example 1 above were loaded into a fluid bed coater (GPCG3, Glatt Air Technique, Ramsey N.J.) and heated at between about 26 to 36° C. for about five minutes. The preheated cores were then coated with an aqueous suspension of methacrylic acid co-polymer type C, White for a total weight gain of 30%. Following coating with methacrylic acid co-polymer type C, the beads are optionally cured by drying in an oven at 50° C. for 48 hours.

Alternatively, for comparison purposes, a portion of the beads were cured under two additional conditions. One portion of beads were cured at 40° C. and 75% RH for 24 hours and a second portion of beads were cured at 40° C. and 75% RH for 24 hours, followed by storage at 40° C. and 75% RH for 3 months.

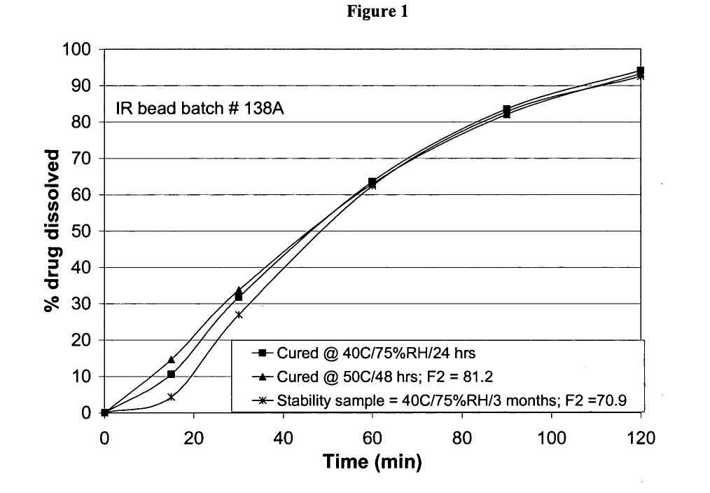

Following curing, the modified release beads of the present example were subjected to dissolution analysis. Dissolution analysis was carried out via the USP I basket method, in 900 ml, aqueous buffer solution pH 5.6, for 120 minutes at 37° C., 100 RPM. The dissolution results for Type I beads cured under different conditions are set forth in Table 3 below and are depicted in FIG. 1. The dissolution profiles for Type I beads cured under different conditions were compared using a model independent statistical approach and the similarity factor, F2. F2 values of 50 or greater ensure equivalence of the two curves. F2 values were calculated from the data points as follows:
F2=50×log{[1+(1/n)Σₜ₋₁ⁿ(Rₜ−Tₜ)²]^{-0.5}×100}

t—dissolution time point

n—number of time points tested

Rₜ—reference batch dissolution time (t)

Tₜ—test batch dissolution at time (t)

The observed similarity of the three dissolution profiles depicted in FIG. 1 is supported by the calculated F2 values. Compared to the reference curve (beads cured at 40° C. and 75% humidity for 24 hours), the F2 values for beads cured under alternative conditions were 81.2 and 70.9 respectively.

TABLE 3
Dissolution data for Type I modified release beads

| | | | Type I cured at |
|---|---|---|---|
| | Type I cured | Type I cured at | 40° C./75% RH/3 |
| Time | at 50° C./48 hrs | 40° C./75% RH/24 hrs | months |
| 0 | 0 | 0 | 0 |
| 15 | 14.6 | 10.6 | 4.3 |
| 30 | 33.7 | 31.8 | 26.9 |
| 60 | 62.9 | 63.5 | 62.4 |
| 120 | 93.3 | 94.1 | 92.5 |

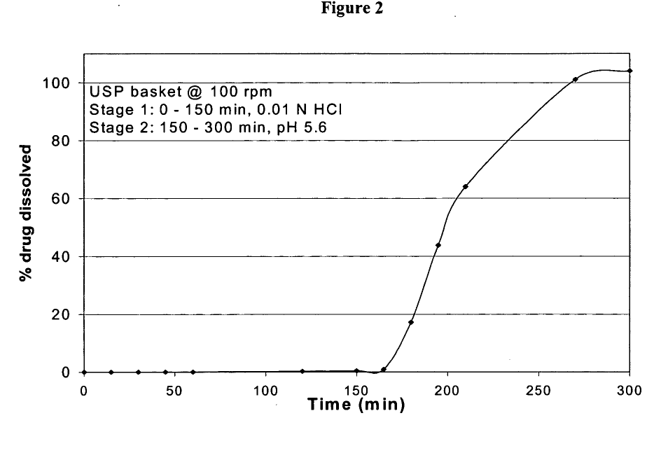

Modified release beads of the present example, cured at 50° C. for 48 hours were also subjected to a modified two phase dissolution analysis. For both phases the dissolution analysis was carried out using USP I basket method, in 900 ml, aqueous buffer solution at 37° C., at 100 RPM The first phase was performed under conditions of 0.1 N HCl containing Polysorbate 80 for 150 minutes. The second phase was carried out at a pH of 5.6 with Polysorbate 1%, for an additional 150 minutes. The dissolution results are shown in FIG. 2. From the FIG. 2, it is evident that amount of lercanidipine dissolved during phase I of the analysis was insubstantial, while a significant amount of lercanidipine was dissolved during phase 2 (i.e., 64% dissolved after 60 minutes in phase 2). The dissolution of lercanidipine in phase 2, conformed to the desired pulsatile pH dependent release profile of about 70% of the lercanidipine, by weight, dissolved after 150 minutes at pH 5.6.

### Example 3

### Preparation of Type II pH Dependent Pulsatile Release Beads

The present example describes the composition and manufacture of a lercanidipine pH dependent pulsatile release bead in which a mixture of Eudragit® L100 and Eudragrit®S100 was applied as an outer coating member to the immediate release core described in Example 1. The composition of the modified release bead of the present Example is shown in Table 4 below. All weights are provided on the basis of the mass of the dried bead composition.

TABLE 4
Type II, lercanidipine pH dependent pulsatile release bead

| Ingredient | mg/g | Weight % Composition |
|---|---|---|
| Lercanidipine immediate | 163.2 | 80.1 |
| release core (122.55 mg/g) | | |
| methacrylic acid copolymer, | 13.53 | 6.6 |
| Type B (Eudragit ® L100) | | |
| methacrylic acid copolymer, | 6.76 | 3.3 |
| Type B (Eudragit ® S100) | | |
| Tiethyl Citrate | 10.15 | 5.0 |
| Talc, USP | 10.15 | 5.0 |
| Ammonia (1.7% solution), NF | 0.21 | 0.1 |

A fraction of the immediate release cores, prepared as described in Example 1 above were loaded into a fluid bed coater (GPCG3, Glatt Air Technique, Ramsey, N.J.) and heated at between about 26 and 36° C. for about five minutes. The preheated cores were then coated with an aqueous suspension of a 1:2 mixture of methacrylic acid copolymer, Type B (Eudragit® S100): methacrylic acid copolymer, Type A (Eudragit® L100) prepared as follows:
1. The methacrylic acid copolymer, Type B and methacrylic acid copolymer, Type A dispersions were separately prepared. To each, add ammonia solution slowly with mixing. Then add triethyl citrate, slowly with continued mixing.
2. Combine both dispersions.
3. Combine the prepared talc dispersion to the polymer dispersion.

The immediate release cores are coated to a weight gain of 25% w/w with the Eudragit® S100:Eudragit® L100 suspension. Following coating with Eudragit® S100:Eudragit® L100, the beads are optionally cured by drying in an oven at 50 for 48° C. hours.

Alternatively, for comparison purposes portions of the beads were cured under two additional conditions. One portion of beads were cured at 40° C. and 75% RH for 24 hours and a second portion of beads were cured at 40° C. and 75% RH for 24 hours, followed by storage at 40° C. and 75% RH for 1 months.

Following curing, the modified release beads of the present example were subjected to dissolution analysis. Dissolution analysis was carried out via the USP I basket method, in 900 ml, aqueous buffer solution pH 6.8, for 120 minutes at 37° C., 100 RPM. The dissolution results for Type II beads cured under different conditions are set forth in Table 5 below and are depicted in FIG. 2. The dissolution profiles for Type II beads cured under different conditions were compared using a model independent statistical approach and the similarity factor, F2. F2 values of 50 or greater ensure equivalence of the two curves. F2 values were calculated from the data points as follows:
F2=50×log{[1+(1/n)Σₜ₋₁ⁿ(Rₜ−Tₜ)²]^{-0.5}×100}

t—dissolution time point

n—number of time points tested

Rₜ—reference batch dissolution time (t)

Tₜ—test batch dissolution at time (t)

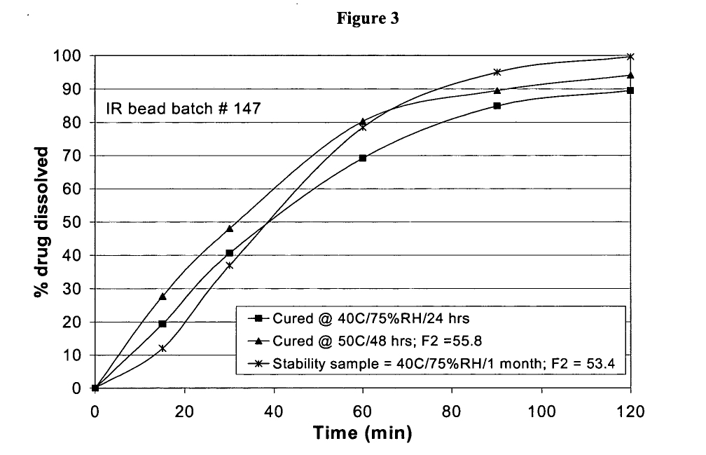

The observed similarity of the three dissolution profiles depicted in FIG. 3 is supported by the calculated F2 values. Compared to the reference curve (beads cured at 40° C. and 75% RH for 24 hours), the F2 values for beads cured under alternative conditions were 55.8 and 53.4 respectively.

TABLE 5
Dissolution data for Type I modified release beads

| | Type II | | Type II cured at |
|---|---|---|---|
| | cured at 50° C./ | Type II cured at | 40° C./75% RH/3 |
| Time | 48 hrs | 40° C./75% RH/24 hrs | months |
| 15 | 27.7 | 19.4 | 12.0 |
| 30 | 48.0 | 40.6 | 36.9 |
| 60 | 80.3 | 69.2 | 78.4 |
| 90 | 89.5 | 84.9 | 95.0 |
| 120 | 94.1 | 89.5 | 99.6 |

Modified release beads of the present example, cured at 50° C. for 48 hours were also subjected to a modified two phase dissolution analysis. For both phases the dissolution analysis was carried out using USP I basket method, in 900 ml, aqueous buffer solution at 37° C., 100 RPM The first phase was performed under routine condition at a pH of 5.6 for 150 minutes. The second phase was carried out at a pH of 6.8 for additional 150 minutes.

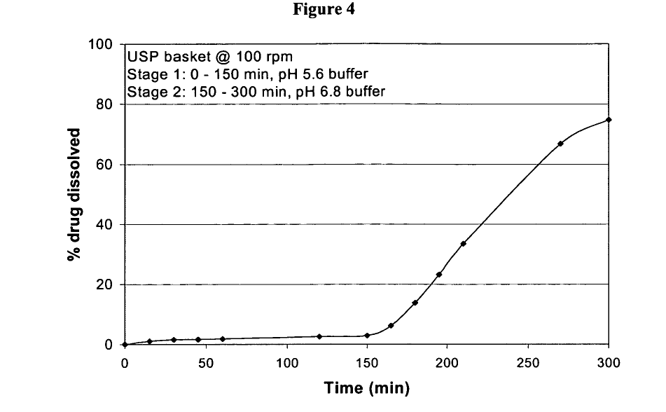

The dissolution results are shown in FIG. 4. From the FIG. 4, it is evident that amount of lercanidipine dissolved at pH 5.6, during phase I of the analysis was insubstantial, while a significant amount of lercanidipine was dissolved during phase II. The dissolution of lercanidipine in phase I conformed to the desired pulsatile dissolution profile of about 70% of the lercanidipine, by weight, dissolved after 150 minutes at pH 6.8 while essentially no release at pH 5.6.

One skilled in the art will appreciate that the above examples show that pH dependent pulsatile lercanidipine dosage forms can be prepared over the entire pH range of gastrointestinal tract. Moreover, the composition can be readily adapted and is not limited to a particular particle coating, tablet coating, granule coating, or capsule coating including hard and soft gelatin containing lercanidipine.

### Example 4

### Preparation of Pulsatile Unit Dosage Form Comprising of Modified Release Lercanidipine Beads

Lercanidipine pH dependent pulsatile release beads, prepared as described in Examples 2 or 3 above, were combined with lercanidipine immediate release beads to form a unit dosage form. Lercanidipine immediate release beads of the present example were prepared having the composition shown in Table 1. The immediate release beads were prepared according to the method described in Example 1 for the preparation of immediate release cores.

TABLE 6
Lercanidipine immediate beads

| Ingredient | mg/g | Weight % Composition |
|---|---|---|
| Lercanidipine HCl | 122.6 | 12.26 |
| Polysorbate 80, NF | 9.2 | 0.92 |
| Sugar Spheres, USP | 818 | 81.8 |
| Opadry Clear (Binder) | 30.6 | 3.06 |
| Opadry Clear (Film | 19.6 | 1.96 |
| Coating) | | |

Two separate unit dosage forms delivering two pulses were prepared, one containing Type I modified release beads (prepared as described in Example 2) and another containing Type II modified release beads (prepared as described in Example 3). The two unit dosage forms, termed Prototype I and Prototype II were prepared as described in Table 7 below.

TABLE 7

| | Type | | |
|---|---|---|---|
| | of modified | Amount of modified | Amount of immediate |
| Prototype | release bead | release bead (mg) | release bead (mg) |
| I | Type I | 20 | 10 |
| II | Type II | 20 | 10 |

Both prototype I and II were subjected to a modified two phase dissolution analysis. For both phases the dissolution analysis was carried out using USP I basket method, in 900 ml, aqueous buffer solution at 37° C., 100 RPM For both prototypes the first phase was performed under conditions of 0.1 N HCl for 150 minutes. For prototype I the second phase was carried out at a pH of 5.6 for an additional 150 minutes, while for prototype II the second phase was carried out at a pH of 5.6 and the third phase was carried out at pH 6.8 for an additional 150 minutes.

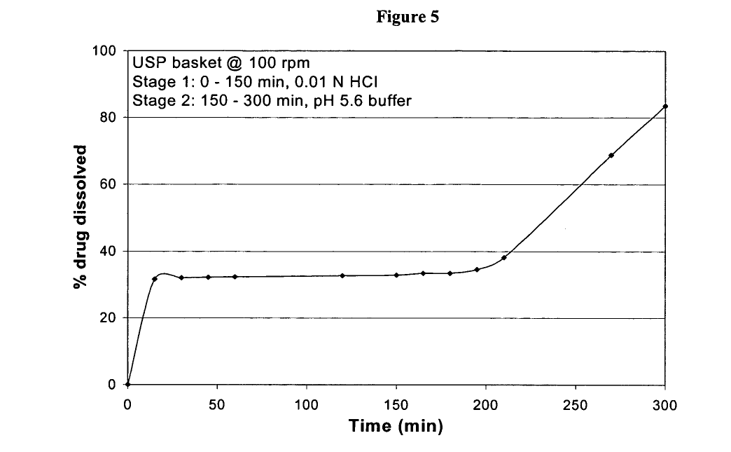

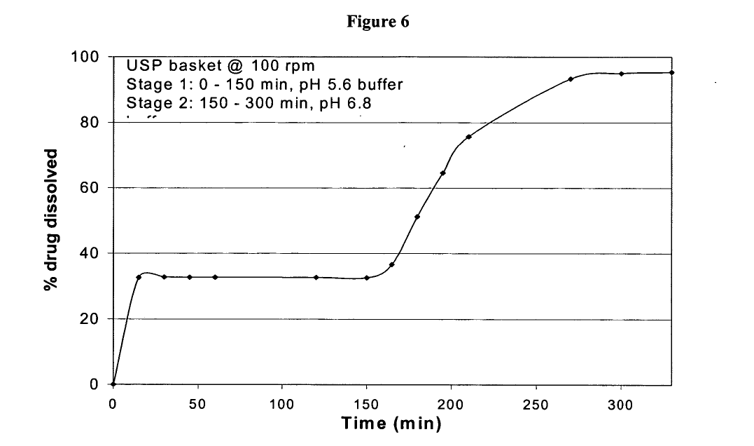

The results of the dissolution analysis for prototypes I and II are shown in FIGS. 5 and 6, respectively. For both prototypes I and II the immediate release portion of the dosage form dissolved almost immediately during phase 1 of the dissolution analysis, with about 80% of the lercanidipine present in the first pulse is released after about 30 minutes. This represent the first pulse delivered in gastric pH.

The level of dissolved lercanidipine remained substantially constant until the second dissolution phase at 150 minutes. This correlated with Gastro-intestinal Transit time of 30 to minutes to about 2 hours. This shows that drug is not leaching from both types the modified release beads at pH of 1.2 simulating stomach pH.

During phase II of the dissolution analysis, about 80% of the lercanidipine was pulsatile released in prototype I at pH 5.6 in less than about 3 hours of phase I with a rapid burst following modified solubilization of pH dependent release of coating of methacrylic acid co-polymer type C. This represents release of lercanidipine through out the small intestine's pH of about 5.5 to 7.

During phase II of the dissolution analysis, negligible amount less than 10% of the lercanidipine is released in prototype II at pH 5.6 in 150 minutes. This represent that drug is not released in ileum portion of small intestine. During the Phase III, drug is released with a rapid burst following modified solubilization of pH dependent release of coating of methacrylic acid co-polymer type A and B. This represents release of lercanidipine through out the small intestine's pH of above about 6.5 to the colonic region

### Example 5

### Dosage Forms Comprising Multiple pH Dependent Pulses

Lercanidipine pH dependent pulsatile release beads, prepared as described as described in Examples 2 or 3 above, were combined with lercanidipine immediate release beads in a gelatin capsule to form a unit dosage form. Lercanidipine immediate release beads of the present example were prepared having the composition shown in Table 6 above. The immediate release beads were prepared according to the method described in Example 1 for the preparation of immediate release cores.

Modified release beads of the present Example were prepared by preheating sugar spheres for about 5 minutes at about 34-44° C. in a fluidized bed coater. The sugar spheres were then coated with an aqueous suspension containing lercanidipine HCl, Opadry™ Clear and Polysorbate 80. The drug loaded beads were then coated with Opadry™ Clear and dried in a fluidized bed for about 5 minutes at about 34-44° C. The dried beads were divided into two portions. One portion of the beads were coated with Acryl-Eze® to the target weight gain, followed by drying in fluidized bed coater for about 5 minutes and cured in an oven at 50° C. for about 48 hours. A second portion of the beads were coated with a Eudragit® S100:Eudragit® L100 coating dispersion to the target weight gain, followed by drying in fluidized bed coater for about 5 minutes and cured in an oven at 50° C. for about 48 hours. Gelatin capsules were then filled with a combination of each of three types of beads representing three different pH dependent pulses. The composition of the pH dependent pulsatile release beads for use in the present example are described in Table 8, below.

### TABLE 8

Composition of Type II, lercanidipine three pH dependent Pulses modified release bead

| | Prototype VII | Prototype IX |
|---|---|---|
| Ingredient | (mg/g) | (mg/g) |
| Lercanidipine HCl | 91.36 | 92.08 |
| Polysorbate 80, NF | 29.71 | 29.22 |
| Sugar spheres, USP | 609.88 | 614.64 |
| Opadry ™ Clear | 68.98 | 68.53 |
| methacrylic acid co-polymer | 37.28 | 36.47 |
| type C (Acryl-Eze ® | | |
| Titanium Dioxide, USP | 13.98 | 13.68 |
| Colloidal Anhydrous Silica, | 1.16 | 1.14 |
| NF | | |
| Sodium Bicarbonate, USP | 1.12 | 1.09 |
| Sodium Lauryl Sulfate, NF | 0.47 | 0.46 |
| methacrylic acid co-polymer | 35.41 | 34.57 |
| type B Eudragit ® S100 | | |
| methacrylic acid co-polymer | 17.69 | 17.27 |
| type A Eudragit ® L100 | | |
| Tiethyl Citrate, PG/NF | 31.05 | 30.33 |
| Talc, USP | 61.29 | 59.91 |
| Ammonia (1.7% solution), NF | 0.55 | 0.54 |

The unit dosage forms of the present example were subjected to a three phase dissolution analysis. For all three phases the dissolution analysis was carried out using USP I basket method, in 900 ml, aqueous buffer solution at 37° C., 100 RPM The first phase was performed under conditions of 0.1 N HCl for 60 minutes. The second phase was carried out at a pH of 5.6 containing Polysorbate 80 for an additional 150 minutes, and the third phase was carried out at a pH of 6.8 containing Polysorbate 80 for an additional 150 minutes.

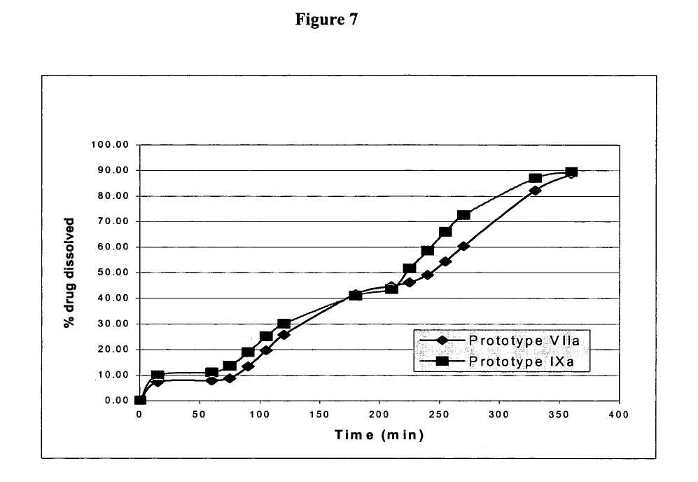

The results of the multiple phase pulsatile pH dependent dissolution analysis for prototypes VII and IX are shown in FIG. 7.

For both prototypes VII and IX, the only the immediate release portion of the dosage form i.e. 10% of the total dose composition, dissolved immediately during phase I of the dissolution analysis.

During the second phase of the dissolution analysis, about 50% of the lercanidipine representing the second pulse was released from both prototypes. At this pH, there was essentially no contribution from the third pulse.

The third Phase, about 80% of the lercanidipine was released representing additive effect of the third pulse from both prototypes released in about 6 hours. The pulsatile release of lercanidipine in multiple pulses corresponds to GI Transit time of about 6 to 8 hours.

### Example 6

### Pharmacokinetics of Unit Dosage Forms Containing Lercanidipine Immediate and Modified Release Beads

The pharmacokinetics of unit dosage forms containing lercanidipine immediate and modified release beads were determined in healthy human subjects following oral administration after a high fat meal.

Experiment

Subjects received the following treatments in sequential order separated by a seven-day washout period:
Treatment A: On Days 1-7 subjects received a 30 mg dose of lercanidipine (5 mg IR, 25 mg Type I).
Treatment B: On Day 14, subjects received a 30 mg dose of lercanidipine (2.5 mg IR, 45 mg Type I), and on Days 15-20, subjects received 60 mg (5 mg IR, 45 mg Type I) daily doses of lercanidipine.
Treatment C: On Day 27, subjects received a 30 mg dose of lercanidipine (2.5 mg IR, 12.5 mg Type 1,15 mg Type II), and on Days 28-33, subjects received 60 mg (5 mg IR, 25 mg Type 1,39 mg Type II) daily doses of lercanidipine.

All dosing occurred 30 minutes after a high fat breakfast. On the second and seventh days of each treatment regimen, blood samples were drawn at the following times for determination of S-lercanidipine plasma concentrations: pre-dose, 0.5, 1, 2, 2.5, 3, 3.5, 4, 4.5, 5, 5.5, 6, 7, 8, 10, 12, 14, 16, 20 and 24 hours past dose.

Results

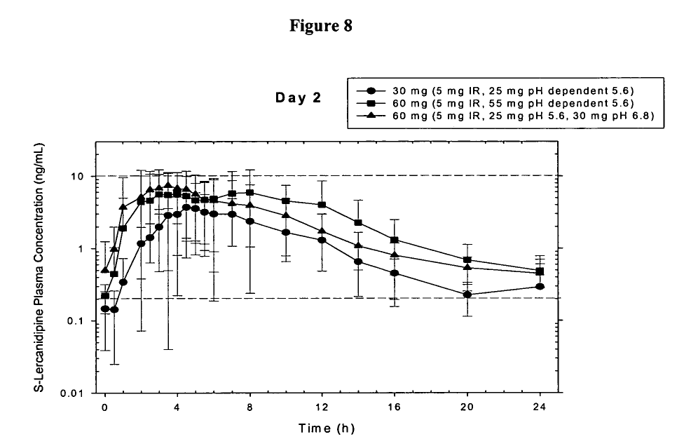

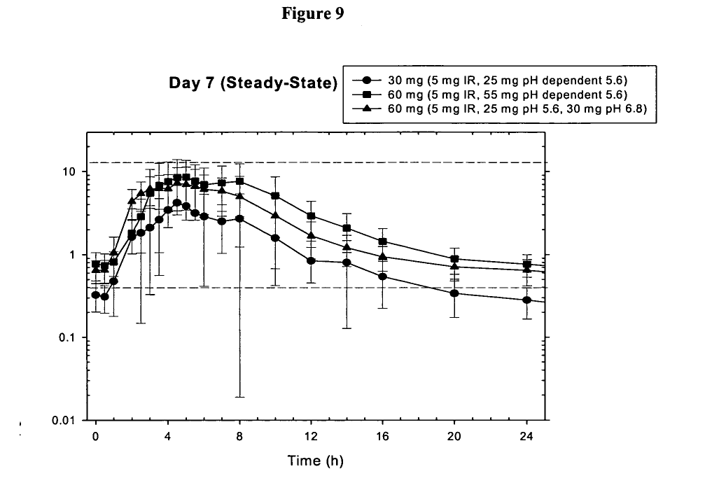

The S-lercanidipine pharmacokinetic parameters on the second an seventh days of dosing are shown in Tables 9 and 10/FIGS. 8 and 9, respectively.

TABLE 9
S-lercanidipine pharmacokinetic parameters (mean ± SD)
on the second day of dosing for unit dosage forms containing immediate
and modified release lercanidipine beads

| | | | 60 mg |
|---|---|---|---|
| | | 60 mg | (5 mg IR, 25 mg |
| | 30 mg (5 mg IR, | (5 mg IR, 55 mg | Type I, 30 mg |
| | 25 mg Type I) | Type I) | Type II) |
| Parameter | (n = 14) | (n = 14) | (n = 14) |
| Cₘₐₓ (ng/mL) | 5.1 ± 2.4 | 12.6 ± 8.3 | 10.6 ± 6.3 |
| Tₘₐₓ (h)* | 5.0 (3.5-12) | 5.5 (2-12) | 3.5 (1-7) |
| AUC₀₋₂₄ | 30.0 ± 12.6 | 70.4 ± 33.6 | 59.3 ± 27.1 |
| (ng · h/mL) | | | |
| AUC_{0-∞} | 36.5 ± 27.2 | 74.4 ± 35.2 | 64.4 ± 27.8 |
| (ng · h/mL) | | | |
| T_{1/2} (h) | 6.7 ± 5.5 | 5.89 ± 2.0 | 8.5 ± 4.4 |

*Median (Range)

TABLE 10
S-lercanidipine pharmacokinetic parameters (mean ± SD) on
the seventh day of dosing for unit dosage forms containing immediate
and modified release lercanidipine beads

| | | | 60 mg |
|---|---|---|---|
| | | 60 mg | (5 mg IR, 25 mg |
| | 30 mg (5 mg IR, | (5 mg IR, 55 mg | Type I, 30 mg |
| | 25 mg Type I) | Type I) | Type II) |
| Parameter | (n = 14) | (n = 14) | (n = 14) |
| Cₘₐₓ (ng/mL) | 5.9 ± 4.6 | 12.7 ± 4.8 | 9.7 ± 4.5 |
| Tₘₐₓ (h)* | 4.5 (2.5-14) | 5 (3-10) | 4.5 (2-8) |
| AUC₀₋₂₄ | 31.9 ± 14.9 | 80.4 ± 23.6 | 63.7 ± 22.0 |
| (ng · h/mL) | | | |
| AUC_{0-∞} | 38.9 ± 18.4 | 100 ± 30.8 | 85.1 ± 30.6 |
| (ng · h/mL) | | | |
| T_{1/2} (h) | 14.7 ± 6.1 | 17.0 ± 4.4 | 21.2 ± 6.3 |

*Median (Range)

Multiple-dose oral administration of the dosage forms of lercanidipine tested in this study at doses of either 30 or 60 mg after a high fat breakfast was safe and well-tolerated in healthy subjects. Treatment B resulted in the highest mean plasma concentrations of S-lercanidipine 24 hours after dosing, and also had the highest mean Cₘₐₓ. In addition, the steady-state AUC₀₋₂₄ₕ was approximately 26 percent greater for Treatment B than Treatment C, suggesting that absorption of Treatment B may be more efficient. For all three treatments, AUC₀₋₂₄ₕ was approximately 1.1 times greater on the seventh day of dosing compared to the second day, indicating that the amount of accumulation of S lercanidipine is low.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A pulsatile release solid dosage form comprising lercanidipine, wherein upon entry of the dosage form to an use environment more than about 80% of the lercanidipine is released in vitro at a pH about 5 to 7.5 within about the first 6 hours and wherein the average Tₘₐₓ is within the range from about 2 hour to about 8 hours.

2. The pulsatile release solid dosage form of claim 1 wherein more than about 70% of the lercanidipine is released in vitro at a pH about 5 to 7.5 within about the first 3 hours.

3. The pulsatile release solid dosage form of claim 1 wherein more than about 50% of the lercanidipine is released in vitro at a pH about 5 to 7.5 within about the first 2 hours.

4. The pulsatile release solid dosage form of claim 1 wherein not more then 20% of the lercanidipine is released in vitro at a pH about 1 to 4.5 within about the first 2 hours following entry of the dosage form into an use environment.

5. The pulsatile release solid dosage form of claim 1 wherein Tₘₐₓ is achieved within the range from about 2 hours to about 7 hours following entry of the dosage form into an use environment.

6. The pulsatile release solid dosage form of claim 1, wherein lercanidipine is lercanidipine hydrochloride.

7. The pulsatile release solid dosage form of claim 1, wherein lercanidipine is present in amounts ranging from about 2 mg to about 60 mg per unit dose.

8. The pulsatile release solid oral dosage form according to claim 1, wherein the modified release solid dosage form is administered to a mammal in need thereof.

9. The pulsatile release solid oral dosage form according to claim 8, wherein the mammal is a human.

10. A method of treating hypertension in a patient in need thereof comprising administering the pulsatile release dosage form of claim 1.

11. The method of claim 10, wherein administration of the pulsatile release dosage form of claim 1 to a patient in need thereof results in an average maximum plasma concentration of lercanidipine is from about 0.5 to about 10 ng/ml, per 20 mg dose of lercanidipine.

12. The pulsatile release solid dosage form according to claim 1, wherein the solid dosage form is encapsulated within a capsule.

13. The pulsatile release solid oral dosage form according to claim 1, wherein the solid dosage form is compressed into a tablet.

14. A pulsatile release pharmaceutical composition comprising:
(1) a core comprising of at least lercanidipine, and optionally, a second layer comprising a film coating;
(2) an outer-most layer comprising at least one pH dependent release modifying polymer; and
(3) optionally, a second layer comprising a film coating.
wherein the pharmaceutical composition has an in vitro dissolution profile such that about 80% of the lercanidipine is released within about the first 6 hours following entry of the form into an use environment, and

15. The pharmaceutical composition according to claim 14, wherein the pharmaceutical composition releases in vitro lercanidipine at a rate of more than about 80% within the first 3 hours following entry of the pharmaceutical composition into an use environment.

16. The pharmaceutical composition according to claim 14, wherein the pharmaceutical composition releases in vitro the lercanidipine at a rate of more than about 80% within the first hour following entry of the pharmaceutical composition into an use environment.

17. The pharmaceutical composition according to claim 14, wherein the outer most layer comprises at least one material selected from the group consisting of an anionic acrylic co-polymer comprising methacrylic acid and methylmethacrylate monomers, cellulose acetatephalate, and Aquacoat.

18. The pharmaceutical composition according to claim 14, wherein the outer most layer is at least 5% of the weight of the core.

19. The pharmaceutical composition according to claim 14, wherein the outer most layer comprises methacrylic acid co-polymer Type C.

20. The pharmaceutical composition according to claim 19, wherein the methacrylic acid co-polymer Type C is sufficient to modify the release of the lercanidipine, such that more than 50% the lercanidipine is released within about a one hour period following exposure of the pharmaceutical composition to an aqueous solution having a pH greater than about 5.6.

21. The pharmaceutical composition according to claim 19, wherein the methacrylic acid co-polymer Type C is sufficient to modify the release of the lercanidipine, such that more than 70% the lercanidipine is released within about a four hour period following exposure of the pharmaceutical composition to an aqueous solution having a pH greater than about 5.6.

22. The pharmaceutical composition according to claim 19, wherein the methacrylic acid co-polymer Type C is sufficient to modify the release of the lercanidipine, such that less than 20% the lercanidipine is released within about a two hour period following exposure of the pharmaceutical composition to an aqueous solution having a pH less than about 4.5

23. The pharmaceutical composition according to claim 19, wherein the methacrylic acid co-polymer Type C is sufficient to modify the release of the lercanidipine, such that less than 10% the lercanidipine is released within about a two hour period following exposure of the pharmaceutical composition to an aqueous solution having a pH between 1 and 2.

24. The pharmaceutical composition according to claim 14, wherein the outer most layer comprises a combination of methacrylic acid co-polymer Type A and methacrylic acid co-polymer Type B.

25. The pharmaceutical composition according to claim 24, wherein the combination of methacrylic acid co-polymer Type A and methacrylic acid co-polymer Type B is sufficient to modify the release of the lercanidipine, such that less than 10% of the lercanidipine is released within about a two hour period following exposure of the pharmaceutical composition to an aqueous solution having a pH between 1 and 5.6

26. The pharmaceutical composition according to claim 24, wherein the combination of methacrylic acid co-polymer Type A to methacrylic acid co-polymer Type B is sufficient to modify the release of the lercanidipine, such that more than about 70% of the lercanidipine is released within about a three hour period following exposure of the pharmaceutical composition to an aqueous solution having a pH greater than about 6.8.

27. The pharmaceutical composition according to claim 24, wherein the combination of methacrylic acid co-polymer Type A to methacrylic acid co-polymer Type B is sufficient to modify the release of the lercanidipine, such that more than about 50% of the lercanidipine is released within about a one hour period following exposure of the pharmaceutical composition to an aqueous solution having a pH greater than about 6.8.

28. The pharmaceutical composition according to claim 24, wherein the weight ratio of methacrylic acid co-polymer Type A to methacrylic acid co-polymer Type B is about 1:2.

29. The pharmaceutical composition according to claim 14, wherein, optionally, the outer most layer further comprises at least one of compounds selected from the group consisting of hydroxypropylmethyl-cellulose, ethyl cellulose, methacrylic acid co-polymer film coating.

30. An oral dosage form comprising:
(i) a plurality of immediate release lercanidipine beads, and
(ii) a plurality of pH dependent pulsatile release lercanidipine beads,
wherein the ratio by mass of (i) to (ii) is from about 1:1 to about 1:5.

31. The solid oral dosage form according to claim 30, wherein the solid oral dosage form is suitable for once daily oral administration.

32. The solid oral dosage form according to claim 30, wherein the solid oral dosage form is suitable for twice daily oral administration.

33. The solid oral dosage form according to claim 30, wherein the total dosage of the lercanidipine is from about 1 to about 80 mg per dose.

34. The solid oral dosage form according to claim 30, wherein the amount of lercanidipine present in the immediate release lercanidipine dosage form is from about 1 to about 20 mg and the amount of lercanidipine present in the pH dependent pulsatile release dosage form is from about 1 to about 80 mg.

35. The solid dosage form according to claim 30, wherein upon administration of the dosage form to a patient, the immediate release lercanidipine is released at the pH of the stomach and provides for a rapid increase in the plasma concentration of lercanidipine, and wherein the pH dependent pulsatile release dosage forms are released at the pH of the small intestine and provide for modified release of the lercanidipine at therapeutic plasma concentrations.

36. The solid oral dosage form according to claim 30, wherein the release of the immediate release lercanidipine results in a maximum in vivo plasma concentration of lercanidipine from about 8 to about 12 ng/ml, within a period of about 1 to about 3 hours following administration of the dosage form to a human, per 20 mg dose of lercanidipine.

37. The solid oral dosage form according to claim 29, wherein administration of the pH dependent pulsatile release dosage form results in a minimum in vivo plasma concentration of lercanidipine from about 0.1 ng/ml to about 0.4 ng/ml for a period from about 18 to about 36 hours following administration of the dosage form to a human, per 20 mg dose of lercanidipine.

38. A pH dependent pulsatile release pharmaceutical composition comprising:
(1) an immediate release core comprising,
(a) an inert core,
(b) a first layer substantially enveloping the inert core, wherein the first layer comprises (i) lercanidipine, (ii) a surfactant, (iii) a binder, and
(c) optionally, a second layer comprising a film coating; and
(2) an outer-most layer comprising at least one pH dependent release modifying polymer,
wherein, upon exposure of the pH dependent pulsatile release lercanidipine composition to an aqueous environment having a pH greater then that of gastric fluid, from about 30 to 40% of the lercanidipine is dissolved within about 1 hour, at least from about 50 to 60% of the lercanidipine is dissolved within about 4 hours, and at least from about 90 to 95% dissolved within about 6 hours.

39. The pH dependent pulsatile release lercanidipine composition of claim 38, wherein the lercanidipine is present in an amount from about 0.001 to about 0.2 mg per gram of the composition.

40. The pH dependent pulsatile release lercanidipine composition of claim 38, wherein the pH dependent release modifying polymer comprises one or more anionic acrylic co-polymers selected from the group consisting of methacrylic acid and methylmethacrylate monomers.

41. The pH dependent pulsatile release lercanidipine composition of claim 40, wherein the outer most layer comprises a pH dependent release modifying polymer selected from a group consisting of Eudragit-L®, Eudragit-S® and Acryl-Eze® and combinations thereof.

42. The pH dependent pulsatile release lercanidipine composition of claim 41, wherein the outer most layer comprises a combination of Eudragit-L® and Eudragit-S®.

43. The pH dependent pulsatile release lercanidipine composition of claim 42, wherein the ratio of Eudragit-L® to Eudragit-S® is sufficient to modify the release of the lercanidipine, such that from about 60 to about 70% of the lercanidipine is dissolved within about an one hour period following exposure of the composition to an aqueous solution having a pH greater than about 6.8.

44. The pH dependent pulsatile release lercanidipine composition of claim 42, wherein the weight ratio of Eudragit-L® to Eudragit-S® is about 1:2.

45. The pH dependent pulsatile release lercanidipine composition of claim 41, wherein the outer most layer comprises Acryl-Eze®.

46. The pH dependent pulsatile release lercanidipine composition of claim 41, wherein the Acryl-Eze® is sufficient to modify the release of the lercanidipine, such that from about 60 to about 70% of the lercanidipine is dissolved within about an one hour period following exposure of the composition to an aqueous solution having a pH greater than about 5.6.

47. The pH dependent pulsatile release lercanidipine composition of claim 38, wherein the outer most layer further comprises a hydroxypropylmethyl-cellulose film coating.

48. The pH dependent pulsatile release lercanidipine composition of claim 47, wherein the film coating is Acryl-Eze®.

49. A pH dependent pulsatile release lercanidipine composition comprising:
(1) an immediate release core comprising,
(a) an inert core,
(b) a first layer substantially enveloping the inert core, wherein the first layer comprises comprising (i) lercanidipine, (ii) a surfactant, (iii) a binder, and
(c) optionally, a second layer comprising a film coating; and
(2) an outer-most layer comprising at least one pH dependent release modifying polymer, wherein, upon administration of the pH dependent pulsatile release lercanidipine composition to a patient, the in vivo plasma concentration of lercanidipine is from about 0.1 ng/ml to about 0.4 ng/ml at a period from about 20 to about 25 after administration of the composition to a patient.
